(19) United States
(12) Patent Application Publication
(10) Pub. No.: US 2010/0075335 A1
(43) Pub. Date: Mar. 25, 2010

(54) COLORIMETRIC METHOD AND KIT FOR THE DETECTION OF SPECIFIC NUCLEIC ACID SEQUENCES USING METAL NANOPARTICLES FUNCTIONALIZED WITH MODIFIED OLIGONUCLEOTIDES

(75) Inventors: José Ricardo Ramos Franco Tavares, Caparica (PT); Pedro Miguel Ribeiro Viana Baptista, Caparica (PT); Gonçalo Maria Reimão Pinto De França Doria, Lisboa (PT); Alcino Orfeu De Leoão Flores, Oeiras (PT)

Correspondence Address: CERMAK KENEALY VAIDYA & NAKAJIMA LLP, 515 EAST BRADDOCK RD SUITE B, Alexandria, VA, 22314, US

(73) Assignees: STAB VIDA, INVESTIGAÇÃO E SERVIÇOS EM CIÊNCIAS BIO, Oeiras (PT); UNIVERSIDADE NOVA DE LISBOA, Lisboa (PT)

(21) Appl. No.: 12/598,906

(22) PCT Filed: May 2, 2008
(86) PCT No.: PCT/IB2008/051708

§ 371 Date: Dec. 8, 2009

(30) Foreign Application Priority Data
May 4, 2007 (PT) 103730

(51) Int. Cl. C12Q 1/68

(52) U.S. Cl. 435/6

(57) ABSTRACT

The present invention relates to a colorimetric method for the detection of specific nucleic acids sequences, including mutations or single nucleotide polymorphisms within nucleic acid sequences, through the aggregation of nanoparticles functionalized with modified oligonucleotides, induced by an increase of the medium's ionic strength. Another aspect of the present invention relates with the development of a kit based on the method of the present invention, allowing for a quick and easy detection of specific nucleic acids sequences, including mutations or single nucleotide polymorphisms within nucleic acid sequences.

### FIELD OF THE INVENTION

The present invention relates to a new colorimetric method and a kit, based on the same method, for the molecular detection of specific nucleic acids sequences (detection and quantification) and any possible differences in the target sequence (polymorphisms and mutations), using metal nanoparticles functionalized with modified oligonucleotides (henceforth designated as “nanoprobes”) for application in several areas of biotechnology, pharmacy, pharmocogenomics and medicine.

### SUMMARY OF THE INVENTION

The method of the present invention is based on the characterization of the colorimetric changes of a solution containing nanoprobes and nucleic acids, being that the colorimetric changes are related with the presence or absence of a nucleic acid with a complementary sequence to the sequence of the nanoprobe. The colorimetric changes of the described solution can be easily and quickly observed by the naked eye or by using standard colorimetric and/or UV-visible spectroscopy methods.

The afore-mentioned method can be applied as part of a kit for the quick and inexpensive detection of mutations and/or polymorphisms in nucleic acids. The nanoprobes can be functionalized with oligonucleotides complementary to any specific nucleic acid sequence, such as, for example, sequences associated with a disease or an alteration of susceptibility to certain diseases (for example, cancer, diabetes) or with differences in the metabolism of certain xenobiotics, with application in several areas of biotechnology, pharmacy, pharmocogenomics and medicine.

### BACKGROUND OF THE INVENTION

The current routine techniques used in clinical and laboratorial diagnostic for the detection of specific nucleic acid sequences require specialized personnel, due to the high complexity of the employed techniques that call for special training and expensive equipment and can only be performed in a laboratorial environment. So, there is a need for the development of new techniques that are simpler, cheaper, quicker and reliable, with a possibility of portability.

The nucleic acids constitute the genetic material of any living organism, harbouring the specific information that allows a complete characterization of that organism. It is possible to identify specific sequences with relevant information of each living organism: identification of sequences; identification of mutations associated with a disease; pathogenic detection, such as bacteria and virus, etc. [1].

The characteristics of a certain living organism are a result of a complex interaction between the genome and the surrounding environment. The majority of the changes in this interaction, no matter how subtle, originate a change in the genetic expression and, therefore, different behaviours on a certain surrounding environment.

Characterization of Human DNA sequences is very important due to the influence that the genome and any of its changes have in the health of an individual. The most common changes in Humans are single nucleotide variations in a specific sequence, being part of a gene or not. These changes are called polymorphisms, more specifically Single Nucleotide Polymorphisms (SNP). SNPs are only one of many possible types of change to the Human genome. Other changes include, but are not limited to, insertions and/or deletions of one or more nucleotides, duplications, etc.

Nevertheless, the most common polymorphisms found in the human genome are SNPs, accounting for around 68% of all detected changes. For some examples, please consult: Celera's Human RefSNP (www.celera.com), NCBI dbSNP (www.ncbi.nlm.nih.gov/SNP/index.html), Human Genome Variation Database-HGVbase (http://hgvbase.cgb.ki.se), The Human Gene Mutation Database-HGMD (http://archive.uwcm.ac.uk/uwcm/mg/hgmd0.html) and HGBASE—Human Genic Bi-Allelic Sequences (http://hgbase.interactiva.de).

SNPs are spread all over the human genome—at coding regions for proteins, where they can alter the properties and the expression of that protein; at non-coding regions, where they can alter expression parameters and modify essential phenomena in the genetic expression (exon excision—splicing); while others do not give rise to any change in the protein, but can change the stability, maturation and the location of messenger RNA.

Other SNPs can be found distant from coding regions and, like the previous, can affect and modulate the expression of one or more genes located nearby or in genomic regions that interact with the region harbouring these polymorphisms. They can also change the availability of promoters, enhancers and repressors of certain genes [2, 3, 4, 5]. For these reasons, the inter-individual variability has been associated to the individual susceptibility to several multifactorial diseases such as cancer, diabetes, as well as to other changes related to the endogenous metabolism or the metabolism of xenobiotics [6, 7].

The literature describes several molecular methodologies for the characterization of nucleic acid sequences in the detection of pathogenic organisms, such as organisms related to disease where the host is Human: tuberculosis, legionellosis, malaria, etc. [8, 9, 10]. The majority of these applications have a low sensitivity and require time consuming methods for the extraction and purification of the nucleic acid. Others require an enzymatic amplification of DNA sequences through a Polymerase Chain Reaction (PCR) [11] in order to increase the sensitivity of the analytical method being used.

There are several applications of PCR for the direct detection of specific nucleic acid sequences without the need for subsequent analytical methods. However, all of these methods are very complex, require trained personnel and expensive equipment to analyse the results. The majority of the techniques used to characterize nucleic acid sequences are based in the selective and specific hybridization between a small oligonucleotide (probe) and a complementary sequence of the nucleic acid (target).

Presently, fluorescence or radioactive based methods are the most commonly used for the detection of specific sequences by hybridization. However, these techniques are highly expensive and time consuming [12, 13]. Also, hybridization techniques usually require a considerable amount of target for signal acquisition. For this reason, they can only be successfully used after PCR amplification of the target nucleic acid sequence in the sample. The PCR allows for an amplification of the number of target nucleic acid molecules available, reproducing what happens in a cell replication.

New techniques of amplification in real-time (e.g., Real-Time PCR) offer a high level of automatization and a reduction of the time needed for amplification and detection. Using these techniques, it is also possible to quantify nucleic acid sequences. However, apart from the expensive equipment needed and the cost of each test, there is also a huge disadvantage associated with these technologies that restrict their use in a higher number of laboratories—sample handling, since it is necessary to have highly purified samples, and, consequently, highly specialized laboratories and personnel are needed [14, 15].

More recently, nucleic acid chips (integrated circuits) are becoming increasingly popular. The majority of their applications can be found in genetic expression studies, where these chips allow for a high-throughput analysis of several genes in a single sample. Basically this technique is based on the simultaneous hybridization of a high number of sequences, requiring a minimum amount of sample. However, amplification of the target in a sample is still necessary. Additionally, the content of the chip is still an unsolved problem, and it is also an expensive technology since these chips cannot be re-used [16].

Currently, the detection of SNPs and/or mutations is performed using techniques based on restriction enzymes, fluorescent or radioactive labels (Amplification Refractory Mutation System—ARMS, Single Strand Conformation Polymorphism—SSCP, Restriction Fragment Length Polymorphism—RFLP, mass spectroscopy, DNA chips, sequencing, etc.), which usually require the amplification of the target by PCR [17]. These techniques are, usually, complex, time consuming and expensive. Among them, the most relevant are the DNA chips and direct sequencing, the latter being the gold standard in molecular diagnostics.

The DNA chip technology has allowed for the simultaneous high-throughput analysis of different SNPs, but, as described above, its highly expensive equipment and reagents prevents its application in routine analysis, being out of reach for most molecular diagnostic laboratories. Direct sequencing is still an expensive process, requiring specialized equipment and personnel, and that is why it is normally used just to confirm the results obtained by other less expensive techniques (in other words, screening techniques).

Several colorimetric methods have been developed to detect nucleic acids [18, 19, 20]. Some of these methods rely on the optical properties (plasmon resonance) of gold and other metal nanoparticles [21, 22], that are a function of their size and shape. These metal nanoparticles, such as gold, silver or gold-silver alloys, are extremely sensitive to changes in the surrounding medium, presenting a colour variation from red to blue, in case of the gold nanoparticles. The colour variation can be the result of the aggregation of several nanoparticles induced, for example, by the presence of a complementary DNA or RNA target. The colour change is a macroscopic response reflecting the events that are occurring at the nanoscale, where the nucleic acid target can react in a complementary or non-complementary way to the nanoprobe. For each of these reactions there is a different response to the incident light that is absorbed. This way, the hybridization of DNA probes bounded to gold nanoparticles in order to identify specific nucleic acids sequences is a low cost and easy to perform technique, and can become an alternative to conventional methods.

Several methods have been described for the synthesis of noble metal nanoparticles, namely colloidal gold nanoparticles. Among the most used methods, there is the HAuCl₄ reduction by sodium citrate, which is well known by any expert in the art. It is possible to control the size of the resulting nanoparticles so as to achieve an average size between 13 and 17 nm. These nanoparticles are stabilized by citrate molecule, avoiding the proximity and aggregation of the nanoparticles, leading to precipitation. This way the inter-nanoparticle distance is kept constant by the repulsion between each charged citrate molecule that acts as a capping agent. Any alteration of the dielectric characteristics of the medium can modify the charges allowing for nanoparticle proximity and aggregation. Coloidal gold nanoparticles present a red colour due to the intense absorbance band at 525 nm from the surface plasmon resonance (SPR). The aggregation of the nanoparticles induces a change of the SPR band to longer wavelengths (between 600 and 700 nm), resulting in a colour change of the solution containing the nanoparticles from red to blue. Aggregation can be induced by several types of changes in the characteristics of the medium: saline concentration/ionic strength, pH, temperature, etc.

Using colloidal gold nanoparticles synthesized as mentioned above, one can substitute the citrate capping agent with DNA oligonucleotides modified with a thiol group at the 3′ or 5′ end. The DNA molecules bind to the surface of the nanoparticles through the thiol group, which possess a high affinity for gold.

This alteration has been described in 1996 by Mirkin and co-workers [23], where they have functionalized coloidal gold nanoparticles with oligonucleotides at their 3′ and 5′ ends (termed “nanoprobes”). The sequences of these nanoprobes were contiguous and complementary to a target in a tail-to-head conformation and were used to characterize the sequence of the target DNA. When hybridization occurs between the two sequences of the nanoprobes and the target DNA, the nanoparticles from the nanoprobes get close to each other and form a cross-link network, leading to the aggregation of the nanoparticles. This aggregation promotes a change in the colour of the solution from red to blue, making this a colorimetric method for DNA detection. It is also possible to detect a single base mismatch by controlling the temperature of denaturation.

Other methods using a similar approach have been described. Of special interest, is the method of Sato and co-workers that describes a detection method for specific DNA sequences based on the hybridization of a single nanoprobe to the target, followed by a single base extension in order to detect a SNP [24]. The increase of the ionic strength of the solution leads only to the aggregation of nanoprobe-complementary target complexes, with a change of colour to blue. This method is temperature independent and describes a fully complementarity (in size and sequence of oligonucleotides) between the sequence of the nanoprobe and the target DNA.

The method of the present invention uses a different approach, were a single nanoprobe with oligonucleotides attached directly to the gold nanoparticles is used, presenting a red colour in solution. The aggregation of these nanoprobes by an increasing ionic strength, promotes a colour change to blue. The presence of a target DNA sequence fully complementary to the sequence of the nanoprobe prevents this aggregation and the solution remains red. This change of colour between solutions of nanoprobes in the presence or absence of complementary DNA sequences constitutes the molecular basis of the method described in this invention.

This principle has already been successfully applied with a great sensitivity in the detection of Mycobacterium tuberculosis' DNA, the etiological agent for human tuberculosis, in clinical samples, and to detect the presence of specific mRNA sequences, allowing its application in genetic expression studies [20, 25]. The method is highly sensitive as it is, without the need for further increase and amplification of the signal, and is able to discriminate fully complementary from single base mismatch sequences, allowing for extending its application to the detection of mutations and SNPs.

Presently there are several documents referring to different techniques related to this area:

Thus, document CN1321776 describes a method for the detection of biological molecules (i.e. DNA or proteins) using gold grains has a reporter of a specific hybridization. Upon hybridization, the grains are immobilized on a surface to which a reagent is further applied to amplify the reporter's signal. This process is substantially different from the proposed invention, namely in the mode of detection of the biological molecule, in which the hybridization to the nanoprobe is directly observed by the naked eye due to a colorimetric change upon increasing the ionic strength of the medium, while the process described in this document requires a microgravimetric measurement of the results with a crystal quartz microbalance.

Documents WO03033735, WO2004042084 and CN1354258 refer to a process of detection and/or determination of specific nucleic acid sequences through the hybridization in chromatographic stripes, using gold nanoparticles functionalized with oligonucleotides as a reporter. The process does not explore any property related to the colour change of gold nanoparticles for the detection of nucleic acids.

Document JP2004329096 describes a method for testing the complementarity between a nucleic acid immobilized on a substrate surface and a target nucleic acid, using gold nanoparticles as a reporter. This method is very distinct from the present invention by the nature of its structure and detection procedure, namely in the present invention the detection is made in a liquid medium without the need to functionalize any immobilized surfaces and the result of the detection of the biological molecule is directly registered by the naked eye through the colorimetric change that can be observed upon increasing the ionic strength of the medium.

Document US2004185462 describes a chip system based on gold electrodes functionalized with oligonucleotides of different sequences. The application of an electrical current allows to detect and analyse single point mutations and SNPs in double stranded DNA. The use of an electrical current is not considered in the present invention, wherein the detection of mutations and SNP is made through a colorimetric process without the need for extra equipment.

Document JP2004275187 relates to a method for the detection of a target DNA in solution, described as the method of Sato and co-workers [24]. It differs from the present invention by the methodology used, which is simpler here, and is reflected also in the final detection result, which is different and less susceptible to false negatives (i.e. formation of aggregates is observed only when a complete hybridization occurs between a fully complementary target DNA and the nanoprobe, while in the present invention the formation of aggregates is observed only in when there is no complete hybridization of the target DNA to the nanoprobe). The present invention has also the advantage of not being limited to nucleic acid targets with a sequence of the same length as the sequence of the nanoprobe, as requested for the method described in this document. This way, the present invention allows for a more practical and straightforward application to detect nucleic acids of different lengths.

Document US2006222595 describes the production of gold nanoparticles, among other metals, for therapeutic use. It also describes a diagnostic method by magnetic resonance, radio-image, X-ray or infrared, where the nanoparticles can be used as a contrasting agent. The present invention does not consider the use of nanoparticles, or nanoprobes, as contrasting agents. It considers the use of nanoparticles to detect a nucleic acid sequence by colorimetric differentiation.

Document JP2005227154 describes a method to detect a specific gene and single base variations, or other similar variations, in that gene. With that purpose, the method uses an electrode where the gene is immobilized and gold nanoparticles functionalized with only one nucleotide, having a different electrochemical activity for each one of the four bases of the genetic code. These modified nanoparticles are then hybridized sequentially with the immobilized gene and at the same time the electrochemical measurements are registered to detect the variations in the gene. The use of electrochemical means is not considered in the present invention for the specific detection, only the observation of a colorimetric change is considered.

Document US2005208592 relates to a method to detect microorganisms using an electrode that can be covered by gold nanoparticles, and one counter-electrode. The use of electrochemical processes is not considered in the present invention, where the observation of colorimetric changes is enough to perform the detection.

Document US2006029969 describes a method to sequence nucleic acids by Surface Enhanced Raman Spectroscopy (SERS), mediated by gold or silver nanoparticles. The present invention does not use any Raman spectroscopy (i.e. SERS, SERFS, CARS), but only the observation of the colorimetric changes of a solution that are visible by the naked eye.

Document WO2006021091 relates to the detection of pharmacological molecules through the use of impedance spectroscopy. On this process, the gold nanoparticles are covered by DNA, and deposited in a gold electrode such as to obtain signal amplification. The present invention does not use impedance spectroscopy, but only the observation of the colorimetric changes of a solution that are visible by the naked eye.

Document CN1392269 describes a method based on a chip to detect nucleic acids. The process includes the extraction of the target gene, marking the target gene with an antigen and hybridizing it with the chip. Subsequently, the gold nanoparticles modified with antibodies complementary to the antigens used to mark the target gene are applied to the chip. The signal is further amplified by a silver reagent and detected by a CCD camera. This process does not relate to the present invention, since it uses an immobilized system on a chip, and nanoparticles functionalized with antigens, instead of oligonucleotides, and it does not use the colorimetric changes of gold nanoparticles for detection. Instead, it uses the silver reduction properties of the gold nanoparticles.

Document CN1464070 reports a method to detect DNA through the combination of a crystal quartz microbalance and gold nanoparticles. The present invention does not consider the use of a crystal quartz microbalance, only considers colorimetric changes visible by the naked eye.

Document US2003013096 reports a method of DNA detection in which gold nanoparticles are placed on a substrate surface to be further functionalized with a thiol modified oligonucleotide. Subsequently, the detection is preformed using a single stranded DNA modified with a fluorescent molecule. The present invention does not use fluorescent molecules as reporter, only colloidal gold nanoparticles.

Document EP0667398 reports a method to detect DNA through gold nanoprobes and using Raman spectroscopy. A solution containing the target DNA and the gold nanoprobe is submitted to different temperatures to denature and hybridize the target DNA with the nanoprobe. If the target DNA sequence is complementary to the sequence of the nanoprobe, the hybridization between them is successful and the formation of a double helix is carried out at the surface of the nanoprobe; otherwise the single strand DNA of the nanoprobe remains unaltered. Using Raman spectroscopy it is possible to identify if the hybridization occurred or not. The present invention does not consider the use of Raman spectroscopy for the detection of a target DNA, only considers colorimetric changes that are visible by the naked eye.

Document US2002127574 describes a method to detect nucleic acids using one, or more, types of nanoparticles functionalized with oligonucleotides. One part of the method, considers the use of gold nanoparticles functionalized with oligonucleotides with sequences that are complementary and contiguous to the nucleic acid target sequence (nanoprobes). The hybridization of the two nanoprobes with sequences complementary and contiguous to the nucleic acid target, promotes a change in colour due to the induced approximation of the nanoparticles. The present invention differs from this method, since it does not require hybridization of two nanoprobes to detect the target DNA and also because it describes a completely distinct process that originates the colour change associated with the detection. The document also describes a method to detect nucleic acids where oligonucleotides with part of the sequence complementary to the target sequence are immobilized on a surface, and the remaining part of the sequence complementary to the target sequence constitutes the sequence of the oligonucleotides functionalized in the gold nanoparticles (nanoprobes). Hence, the nucleic acid targets to be detected and analysed partly hybridize with the respective immobilized oligonucleotides and, simultaneously, partly with the corresponding gold nanoprobe. This hybridization is also known as sandwich hybridization. The presence of the nanoprobe in certain points of the surface reports the target sequences to be detected. Optionally, the signal can be further amplified by a reduction process using a silver reagent. In this process, and in all of the process variants, the use of colorimetric changes is not considered to detect the target molecule, such as it is in the present invention, which relies on a colorimetric change. The document US2002127574 also describes processes to fabricate nanomaterials and nanostructures, and processes to purify nucleic acids, all of which are not considered in the present invention and are not part of its purpose.

Document WO2006104979 describes a method to identify proteins through a process designated as barcode. Two types of particles are used: magnetic microparticles functionalized with a specific antibody that is complementary to the target protein to be detected; and gold nanoparticles functionalized with specific antibodies that are complementary to the same target protein and a large number of oligonucleotides that are hybridized with their complementary oligonucleotides. These complementary oligonucleotides function as biological barcodes that can be associated to a certain protein. The two types of nanoparticles are mixed in a solution containing the target protein to detect. Both particles bind only to their specific target protein. The complexes of particles formed with the target protein are then captured by a magnetic field, while the remaining proteins and nanoparticles are washed out. Afterwards, the biological barcodes are released from the nanoparticles and analyzed to identify the target protein. The method to analyse the biological barcodes is described on document US2002127574. The present invention does not consider the detection of proteins and differs from the method described in the document US2002127574, since it is based on colorimetric changes to detect and identify the target of interest.

Document WO03048769 describes a method to monitor in real time the amplification by PCR, using gold nanoprobes. The method conjugates the process of amplification by PCR with the detection method based on the cross-linking of two gold nanoprobes. Thus, as the amplicon is produced, the two nanoprobes that are contiguously complementary to the amplicon sequence will hybridize with it resulting in a colour change of the solution that can be measured in real time by a spectrophotometer device. The present invention does not rely on the cross-linking of two nanoprobes to change the colour of the solution in the presence of a DNA target. It depends only on the colorimetric changes of one nanoprobe that are induced by an increasing ionic strength, reflecting the absence of a fully complementary target in solution.

Document CN1661094 refers to a colorimetric method to detect genetic mutations through the combination of amplification of specific alleles, gold nanoprobes and an electrolyte. When a DNA target is present in solution with a mutation complementary to the primer of the allele specific amplification, this primer is consumed in order to achieve a successful amplification and, therefore, the formation of a double strand DNA (solution A). When the target DNA is not present, or is not complementary to the primer, the amplification reaction is not carried out successfully and the single stranded primer remains in solution (solution B). Upon addition of an electrolyte to the solutions, only solution A changes colour from red to blue. In the case of solution B, the initial colour remains unaltered. The described process differs substantially from the present invention since it does not use gold nanoparticles functionalized with modified oligonucleotides (nanoprobes). On the other hand, the colorimetric result differs from the present invention, since on a positive result (i.e. detection of a target DNA complementary to the primer) a colour change is observed, while in the present invention the colour does not change in the presence of a fully complementary target. The process described in this document is also dependent of an amplification reaction of specific alleles, while in the present invention, such amplification reaction is an optional step.

Tests for biological samples, such as DNA/RNA and proteins, have already been used on a vast field of applications.

There are some kits based on the previously described technologies, different from the proposed method, for detection of mutations/SNPs. Namely, based on fluorescent markers there is the AcycloPrime-FP SNP detection system (Perkin Elmer), the SNaPshot Multiplex system (Applied Biosystems), the SNPlex genotyping system (Applied Biosystems) and the LightTyper system (Roche Applied Science); based on Real-Time PCR there is the TaqMan system (Applied Biosystems); and based on DNA chip technology, the GeneChip Custom SNP system (Affymetrix). However, all of these kits/systems require specialized personnel, expensive reagents and non-portable equipments, preventing its use in the point-of-care.

Thus, a kit based on the colorimetric method of the present invention has the advantage to easily and cheaply detect mutations/SNP in nucleic acids directly at the point-of-care, without the need for specialized personnel and equipment.

### GENERAL DESCRIPTION OF THE INVENTION

The present invention describes a nanotechnology process for the specific and ultra-sensitive detection of mutations/SNPs in nucleic acids sequences, in a simple, quick and inexpensive way, without compromising quality.

The method is based on the utilization of colloidal gold nanoparticles functionalized with oligonucleotides (nanoprobes) for detection of mutations/SNPs in nucleic acids sequences.

The detection mechanism is based on the optical properties of the coloidal gold solution, where the absorbance peak changes upon variation of the ionic strength (salt addition), accordingly to the inter-nanoparticle distance and depending on the presence or absence of a complementary nucleic acid sequence.

The colour change is a macroscopical response reflecting nanoscale phenomena, where the DNA/RNA can react in a complementary or non-complementary way with the oligonucleotide of a known sequence that is functionalized to the nanoparticles. For each one of these reactions there is a different absorbance response to the incident light.

The nucleic acid nanoprobe is constituted by colloidal gold nanoparticles that are linked to a known sequence of DNA. This solution possesses nanoparticles with a mean diameter of 17 nm, or more, in which the absorbance peak is located around 520 nm due to its plasmon resonance, presenting a red colour.

The coloidal gold nanoparticles are synthesized by methods know of any specialist in the art, such as the gold salt (e.g. HAuCl₄) reduction method with a citrate salt. It is possible to control the size of the resulting nanoparticles by altering the citrate/HAuCl₄ proportions in order to synthesize nanoparticles with a mean diameter between 13 and 17 nm.

These nanoparticles are stabilized by the citrate molecules that prevent them from getting close to each other and aggregate, finally precipitating. The inter-particle distance is thus maintained by the repulsion of the citrate ionic charges. A change on the dielectrical characteristics of the surrounding medium can lead to an alteration of these charges in such a way that the nanoparticles can get closer to each other and aggregate.

Solutions containing colloidal gold nanoparticles, with a mean diameter between 13 and 17 nm, present a red colour due to an intense absorbance at 525 nm, resulting from their surface plasmon resonance (SPR).

The aggregation of the nanoparticles induces a shift of the SPR band towards higher wavelengths (ca. 600 to 700 nm), resulting on a colour change from red to blue of the solution containing the nanoparticles. The aggregation can be induced by several types of changes in the surrounding medium: saline concentration and ionic strength, pH, temperature, etc.

The colloidal gold nanoparticles can be functionalized by single strand nucleic acids modified with a thiol group at the 3′ or 5′ end of the DNA molecule. The DNA molecules bind to the nanoparticles surface through the thiol group, which has a high affinity to gold. The single strand DNA (ssDNA) molecule can also have n alkyl groups between the nucleotides and the thiol group. The ssDNA functionalized nanoparticles—nanoprobes—remain stabilized in solution due to the strong repulsion between the negative charges of the phosphate molecules in the DNA, which have substituted the citrate ions and can be, in number, around 200 or more. This way, the nanoprobe solution is red and stable at pH 7.0-8.0, with a characteristic absorbance peak around 520-525 nm.

The aggregation of the nanoprobes by altering the characteristics of the dielectric medium (pH, ionic strength, saline concentration, temperature), induces a red shift towards 600 to 700 nm and the solution becomes blue.

The biological sample containing the target DNA/RNA to be analysed is denatured by heat together with the corresponding nanoprobe. After cooling down, during which the hybridization between the DNA from the nanoprobe and the target DNA/RNA occurs if they are complementary, an electrolyte solution is added to quickly reveal the final result. This electrolyte can be, but is not limited to, NaCl, MgCl₂, NiCl₂, NaBr, ZnCl₂, MnCl₂, BrCl, CdCl₂, CaCl₂, CoCl₂, CoCl₃, CuCl₂, CuCl, PbCl₂, PtCl₂, PtCl₄, KCl, RbCl, AgCl, SnCl₂, BrF, LiBr, KBr, AgBr, NaNO₂, Na₃PO₄, Na₂HPO₄, NaH₂PO₄, KH₂PO₄, K₂HPO₄, and is responsible for the increasing ionic strength of the medium. Thus, if the analysed sample contains a 100% complementary sequence to that of the nanoprobe, the initial red colour will be kept upon the addition of the electrolyte, since the presence of a nucleic acid sequence fully complementary to the nanoprobe leads to a specific hybridization and avoids aggregation of the nanoparticles upon increasing ionic strength, and thus the solution remains red.

In the absence of a complementary sequence, or if there is a single base mismatch to the nanoprobe being used, a colour change from red to blue can be observed since there is no protecting effect as previously described, the nanoprobes aggregate and the solution changes colour to blue.

The colorimetric changes of solutions containing the nanoprobes in presence or absence of a complementary nucleic acid sequence constitute the molecular basis of the method described in this invention. This method is highly sensitive by itself, without the need to further increase or signal amplification, allowing discrimination between fully complementary and single base mismatched sequences. It is then possible to discriminate between fully complementary sequences and sequences with one or more non-complementary nucleotides, due to small changes on stability of the nanoprobes in solution, i.e., at the same ionic strength the resistance of the nanoprobes to aggregation increases with sequence complementarity. The non-complementary nucleotides destabilize the nanoprobe-target complex, decreasing the repulsion between nanoprobes and leading to a slow and gradual aggregation that can be monitored and constitutes the basis for the detection of SNPs and mutations in nucleic acids.

The method consists on adding the sample of nucleic acids to a solution containing the nanoprobe with a final gold nanoparticle concentration of 2.5 nM.

Each test is based on three simultaneous assays—Blank, without the nucleic acids; Negative, with a non-complementary DNA/RNA to the nanoprobe; Positive, with a complementary DNA/RNA to the nanoprobe. These assays (blank, negative, positive) constitute the control of the test.

In parallel to the control assays there can be one (or more) assay(s) with the target nucleic acid to be tested for one (or more) specific mutation(s)/SNPs.

Hybridization is carried out by cooling down to room temperature between 10 to 30° C. after pre-heating to 95° C. to fully denature the DNA double helix or destroy RNA secondary structures. After the cooling period, which can last between 0 minutes to 24 hours, a saline solution is added to change the ionic strength. The concentration of the nucleic acid target can be between 0 and 100 μg/ml, with an optimal concentration around 18 to 36 μg/ml.

Change in the ionic strength can be achieved by adding a saline solution of NaCl, MgCl₂, NiCl₂, NaBr, ZnCl₂, MnCl₂, BrCl, CdCl₂, CaCl₂, CoCl₂, CoCl₃, CuCl₂, CuCl, PbCl₂, PtCl₂, PtCl₄, KCl, RbCl, AgCl, SnCl₂, BrF, LiBr, KBr, AgBr, NaNO₂, Na₃PO₄, Na₂HPO₄, NaH₂PO₄, KH₂PO₄, K₂HPO₄, or other. The change in the hybridization solutions and/or ressuspension of the nanoprobes can also have an effect in the discriminatory aggregation of the nanoprobes. Each one of these changes can also be related to the pH of the medium.

For the same ionic strength of the medium, pH and temperature, the molecules for which the nucleic mismatch is in the 3′ end of the nanoprobe sequence have a higher destabilizing effect when compared to those located in the internal sequence of the nanoprobe.

Besides detection by the naked eye, the colorimetric changes can also be followed by a simple spectrophotometer in order to analyse the shift in the maximum absorbance peak. This method can be analysed in parallel by means of a microplate reader that can determine the absorbance peaks of the samples.

The colorimetric differences of the solution containing the nanoprobes, in presence or absence of a complementary sequence, can be easily and quickly observed by the naked eye without the need for additional instrumentation. This colour change, from red to blue, is due to the changes in the surface plasmon resonance of the gold nanoparticles, characteristic of their shape, size and surrounding medium.

The results show two absorbance maximums with a Gaussian function behaviour, namely: centred at 525 nm, which represents the interaction between the nanoprobe and the fully complementary DNA/RNA; centred at the lower energies, around 600 nm, which represents the non-complementary reactions, where only a single mismatch can lead to aggregation (see FIG. 2).

The method allows a quick diagnostic of mutations and genetic polymorphisms associated with diseases or an alteration of susceptibility to certain diseases (for example, cancer) or to certain xenobiotics (pharmacogenomics).

Noble metal nanoparticles (generally on coloidal solutions) show a great potential to be applied in chemical and biological detection due to their unique optical and electrical properties. Furthermore, the synthesis of these nanoparticles is cheap and easy to produce.

Being a colorimetric method it allows for the results to be quickly obtained without the need for large and expensive equipment, making it possible to be executed outside of a laboratory environment.

The speed, simplicity, portability and specificity of the method, associated with a low cost, are unique and relevant characteristics that potentiate its application in the clinical and medical care environment, as a point-of-care diagnostic method. This process allows for the in situ detection in real time, reducing the time and costs usually associated.

The proposed method has a high potential to quickly become an important and indispensable tool for all health care professionals, among others.

Biological sample analysis, for example in the specific case of DNA and proteins, is used in forensics, in the clinical and laboratorial diagnostic markets and in research. Presently, these techniques are being used to diagnose infect-contagious diseases, though cancer diagnosis and genetics (research) also represent relevant areas of application.

The main industries that presently use methods for the detection of biological samples, such as, for example, in the specific case of DNA/RNA and proteins, are:
Defence Organizations: issues related to biosafety have enhanced the demand for reliable and quick DNA/RNA tests, especially in the United States.
Medical Institutions: they are used for medical diagnostics, namely to track genetic diseases or to identify pathogens.
R&D Organizations: several molecular tests using DNA/RNA detection are performed in research.
Health care centres and laboratories to track and analyse contagious diseases.
World Health Organization
Governmental and Non-Governmental organizations that support the detection, eradication and fight against pathogenic diseases usually associated to poverty (malaria, tuberculosis, HIV)

Apart from these industries, that presently use methods to detect biological samples, such as the specific case of DNA/RNA and proteins, new segments can also make use of these tests, such as:
Food Industry, to control the quality of their products, through the laboratorial analysis of samples of their products.
Pharmaceutical industry, to test and quantify the action of the medical drugs.
Agriculture/veterinary industry, to detect pathogens, and in particular to the agriculture industry, to detect genetically modified organisms.

The proposed method can be reproduced in a device such as in a kit, to detect mutations/SNP in nucleic acids at the point-of-care. This kit is thus composed of one or more solutions with nanoprobes to detect the mutations/SNPs of interest, a hybridization buffer, a development solution and one or more control solutions containing the non-complementary and complementary targets to the nanoprobe(s). These solutions are then mixed in wells, of one or more microplates, following the method's procedure and thus allowing to directly visualize the results by the naked eye, or, optionally, to register the results by UV/visible spectroscopy.

### BRIEF DESCRIPTION OF THE DRAWINGS

FIG. 1—Method Scheme

Each test is based on three assays that constitute the control of the reaction—Blank (A), nanoprobe (1) without nucleic acids; Negative (B), nanoprobe (1) with a known DNA/RNA target non-complementary to the nanoprobe (2); Positive (C), with a known DNA/RNA target complementary to the nanoprobe (3). In parallel to the control assays, one or more assay(s) (D) can be performed with the target nucleic acid (4) being tested for one (or more) specific mutation(s)/SNPs. The hybridization (5) is carried out by heating and cooling down the solutions; at this point no colorimetric changes are observed. After cooling down, a saline solution is added to change the ionic strength (6) and the colorimetric change of each assay is registered by the naked eye, or with the help of a UV/visible spectrophotometer.

FIG. 2—Example of Detection of Single Point Mutations Associated to Beta Thalassaemia

Visible spectrums of the assays, acquired 15 minutes after NaCl addition ([NaCl]=2 M). Nanoprobe with a DNA from a normal HBB gene ((C)—solid line); nanoprobe with a DNA harbouring the single point mutation IVS1, nt1 at the HBB gene ((D1)—slashed line); nanoprobe with a DNA harbouring the single point mutation IVS1, nt2 at the HBB gene ((D2)—slash-dot-slash line); nanoprobe with a DNA harbouring the single point mutation IVS1, nt6 at the HBB gene ((D3)—dotted line). The relative position of each mutation (▾) relative to the nanoprobe can be visualized on the right side of the figure, along with the photographs taken before and 15 minutes after (→) the salt addition.

### DETAILED DESCRIPTION OF THE INVENTION

1. Preparation of the Nanoprobes and Gold Nanoparticles

The colloidal gold nanoparticles are synthesized by methods know of any specialist in the art, such as the gold salt (for ex. HAuCl₄) reduction method with a citrate salt. Briefly, a solution of HAuCl₄ (1 mM; 500 mL) is brought to a boil while stirring vigorously. A solution of sodium citrate (38.8 mM; 50 mL) is then quickly added and the solution's colour changes from light yellow to dark red. After this colour change the mixture is kept refluxing for an additional 15 minutes with continuous stirring, left to cool to room temperature and stored in the dark.

It is possible to control the size of the resulting nanoparticles by altering the citrate/HAuCl₄ proportions in order to synthesize nanoparticles with a mean diameter between 13 and 17 nm.

These nanoparticles are stabilized by the citrate molecules that prevent them from getting close to each other and aggregate, finally precipitating. The colloidal gold nanoparticles solutions, with a mean diameter between 13 and 17 nm, present a red colour due to an intense absorbance at 525 nm, characteristic of their surface plasmon resonance (SPR).

The colloidal gold nanoparticles are functionalized by single strand DNA modified with a thiol group at the 3′ or 5′ end o the DNA molecule. Briefly, a colloidal gold nanoparticles solution with a mean diameter of 13 nm (17 nM in nanoparticles; 5 mL) is functionalized with thiol modified oligonucleotides in a final concentration of 3.61 μM. After a 16 hour resting period, a 50 mM (pH 7) phosphate buffer, 0.5 M NaCl solution is added to a final concentration of 10 mM phosphate, 0.1 M NaCl. After resting for 48 hours, the solution is centrifuged for 25 minutes at 14,000 rpm and the supernatant is discarded. The resulting precipitate is washed with 5 mL of 10 mM (pH 7) phosphate buffer, 0.1 M NaCl, recentrifuged and redispersed in 5 mL of 10 mM (pH 7) phosphate buffer, 0.1 M NaCl, and stored in the dark at 4° C.

The DNA molecules thus bind to the nanoparticles surface through the thiol group, which has a great affinity to gold. The single strand DNA (ssDNA) molecule can also have n alkyl groups between the nucleotides and the thiol group. The ssDNA functionalized nanoparticles—nanoprobes—remain stabilized in solution due to the strong repulsion between the negative charges of the phosphate molecules in the DNA, which have substituted the citrate ions and can be, in number, around 200 or more. This way, the nanoprobe solution is red and stable at pH 7.0-8.0, with a characteristic absorbance peak around 520-525 nm.

2. Preparation of the Target Samples

The samples containing the target nucleic acids can be prepared with any current method known by a specialist in the art, or by using any extraction and purification kit for nucleic acids available in the market. Afterwards, the region of interest can be amplified by PCR, or any other isothermic amplification (ex. LAMP—Loop-mediated isothermal amplification).

3. Nanoprobe Hybridization and Colorimetric Detection

Each test is made of three basic assays—Blank: nanoprobe without the nucleic acids; Negative: nanoprobe with a known non-complementary DNA/RNA to the nanoprobe; Positive: nanoprobe with a known complementary DNA/RNA to the nanoprobe. In parallel to the control assays there can be one (or more) assay(s) with the target nucleic acid to be tested for one (or more) specific mutation(s)/SNPs.

The method consists in adding a sample of the target nucleic acid to a solution of nanoprobe with a 2.5 nM final concentration of gold nanoparticles. The concentration of the target nucleic acid can be between 0 and 100 μg/ml, with an optimal concentration around 18 to 36 μg/ml.

The hybridization is carried out by cooling down to a room temperature between 10 to 30° C. after a pre-heating to fully denature the double helix of the DNA or the secondary structures of RNA in the sample.

After the cooling period, which can last up to 24 hours, a saline solution is added to change the ionic strength. The change in the ionic strength can be achieved by adding a saline solution of NaCl, MgCl₂, NiCl₂, NaBr, ZnCl₂, MnCl₂, BrCl, CdCl₂, CaCl₂, CoCl₂, CoCl₃, CuCl₂, CuCl, PbCl₂, PtCl₂, PtCl₄, KCl, RbCl, AgCl, SnCl₂, BrF, LiBr, KBr, AgBr, NaNO₂, Na₃PO₄, Na₂HPO₄, NaH₂PO₄, KH₂PO₄, K₂HPO₄, or other. The change in the hybridization solutions and/or ressuspension of the nanoprobes can also have an effect in the discriminatory aggregation of the nanoprobes. Each one of these changes can also be related to the pH value of the medium.

Besides detection by the naked eye, the colorimetric changes can also be followed by a simple spectrophotometer in order to analyse the shift in the maximum absorbance peak. This method can be analysed in parallel by means of a microplate reader that can determine the absorbance peaks of the samples.

4. Interpretation of Results

The results show two absorbance maximums with a Gaussian function behaviour, namely: centred at 525 nm, which represents the interaction between the nanoprobe and the fully complementary DNA/RNA; centred at the lower energies, around 600 nm, which represents the non-complementary reactions, where only a single mismatch can lead to aggregation (see FIG. 2).

Thus, if the analysed sample contains a sequence that is 100% complementary to the sequence of the nanoprobe that is being used, the initial red colour will be kept upon the addition of the electrolyte, since the presence of a nucleic acid sequence fully complementary to the nanoprobe leads to a specific hybridization and avoids the aggregation of the nanoparticles upon the increase of the ionic strength, and the solution remains red.

In absence of a complementary sequence, or if there is a single base mismatch to the sequence of the nanoprobe being used, a colour change from red to blue can be observed since there is no protecting effect as the one previously described, and so the nanoprobe aggregates and the solution colour changes to blue.

These colour changes between solutions containing the nanoprobes in presence or absence of a complementary nucleic acid sequence constitute the molecular basis of the method described in this invention. This method is highly sensitive by itself, without the need to further increase or amplify the signal, allowing the discrimination between fully complementary and single base mismatch sequences. It is then possible to discriminate fully complementary sequences from sequences with one or more non-complementary nucleotides, due to small changes on the stability of the nanoprobes in solution, i.e., at the same ionic strength the resistance of the nanoprobes to aggregate is higher if the complementarity between sequences is greater. The non-complementary nucleotides destabilize the nanoprobe-target complex, decreasing the repulsion between nanoprobes and leading to a slow and gradual aggregation that can be monitored, constituting the basis for the detection of SNP and mutations in DNA (see FIG. 2).

5. Kit for the Detection of Mutations/SNP in Nucleic Acids Sequences

The kit is made of one or more solutions with the nanoprobes to detect certain mutations/SNPs of interest, a hybridization buffer, a development solution and one or more control solutions containing target nucleic acids that are non-complementary and complementary to the nanoprobe(s).

The solution containing the nanoprobes can be supplied in wells of one, or more, microplate(s), or in containers for later application in the wells of the microplate(s) and should be stored in the dark at 4° C.

The hybridization buffer can be supplied together with the nanoprobes, or separately in its own container for further application in the wells of the microplate(s) containing the nanoprobe(s).

The remaining development solutions; the control solution containing a target nucleic acid non-complementary to the nanoprobe; and one or more control solutions containing a target nucleic acid complementary to the nanoprobe(s); should by supplied separately in independent recipients.

The samples with the target nucleic acid(s) to be analysed can be prepared as previously described in item 2. of the method—“Preparation of the target samples”.

Each test will be performed in the wells of the microplate(s) as previously described in item 3. of the method—“Nanoprobe hybridization and colorimetric detection”.

Briefly, the nucleic acid(s) sample(s) is (are) added to the mix of nanoprobe(s) and hybridization buffer in the corresponding wells. The microplate is sealed, heated and cooled down to promote hybridization of the target nucleic acid(s) with the nanoprobe(s). Subsequently, the development solution is added to each well of the microplate(s) and the resulting colorimetric change is registered by the naked eye or, optionally, using a microplate reader to perform a UV/visible absorbance spectroscopy analysis.

The interpretation of the results will be made as previously described in item 4. of the method—“Interpretation of results”.

### Examples

The present invention can be further illustrated by the following specific example.

### Example 1

Detection of Single Point Mutations Associated with Beta Thalassaemia

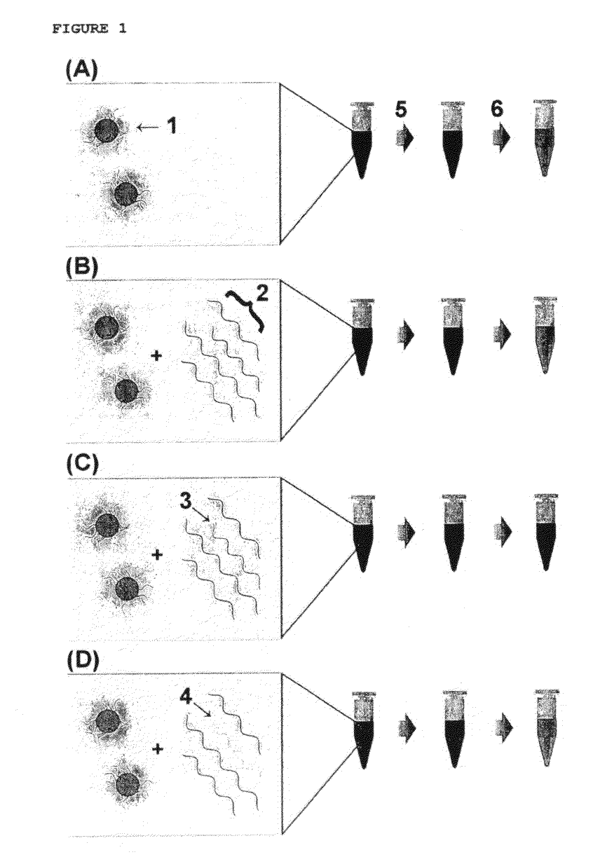

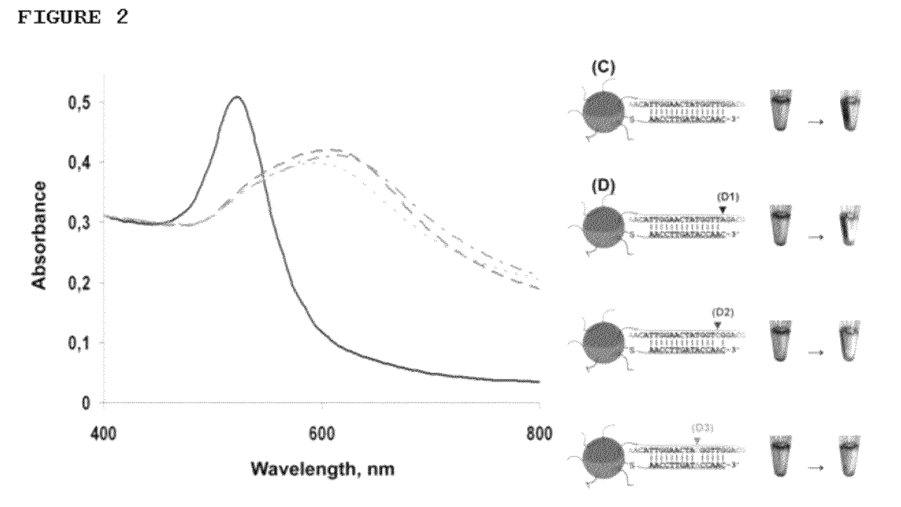

The method has been successfully applied to detect single point mutations within the beta globin gene. Namely, a nanoprobe was synthesized by functionalizing 13 nm gold nanoparticles with a 5′ thiol modified oligonucleotide with a 15 bp sequence harbouring three of the most frequent mutations in the Portuguese and Mediterranean population, associated to beta thalassaemia (IVS1, nt1; IVS1, nt2; IVS1, nt6) [26]. Subsequently, in a total volume of 60 ul, several assays were performed by mixing nanoprobe, in a final concentration of 2.5 nM of gold nanoparticles, with an amplicon harbouring one of the point mutations mentioned above, in a final concentration of 36 μg/mL (see FIG. 1—“D”). The control assays were prepared simultaneously, namely a positive control—with complementary DNA (see FIG. 1—“C”); a negative control—with a non-complementary DNA (see FIG. 1—“B”) and a Blank—replacing the target DNA with 10 mM phosphate buffer (pH 8) (see FIG. 1—“A”). After 10 minutes of denaturation at 95° C., the mixtures were allowed to stand at room temperature for 30 minutes, and then a 5 M NaCl solution was added for a final concentration of 2 M NaCl. After 15 minutes at room temperature the results were registered by photography and UV/visible spectroscopy (see FIG. 2). Observation by the naked eye, or by using UV/visible spectroscopy, allows to conclude that, upon rising the ionic strength of the medium by adding NaCl, the assays in which the target DNA harboured a single point mutation when compared to the nanoprobe sequence had changed colour from red to blue (see FIG. 2—“D”), while the assay with a complementary target DNA (positive control) kept its red colour (see FIG. 2—“C”). Furthermore, a single nanoprobe allowed the detection of three different point mutations (see FIG. 2—“D1”, “D2 and “D3”).

### REFERENCES

[1] Videira, Arnaldo, Engenharia Genética—Principios e Aplicações. LIDEL (2001).

[2] Tabor H K, Risch N J, Myers R M. “Candidate-gene approaches for studying complex genetic traits: practical considerations”, Nature Reviews Genetics, 3:391-397, 2002.

[3] Zhao H, Pfeiffer R, Gail M H. “Haplotype analysis in population genetics and association studies”, Pharmacogenomics, 4:171-178, 2003.

[4] Collins F S, Brooks L D, Chakravarti A. “A DNA polymorphism discovery resource for research on human genetic variation”, Genome Research, 8:1229-1231, 1998.

[5] Wang Z, Moult J. “SNP, protein structure, and disease”, Human Mutation, 17:263-270, 2001.

[6] Hung R J, Hall J, Brennan P, Boffetta P. “Genetic polymorphisms in the base excision repair pathway and cancer risk: a HuGE review”, Am J Epidemiol., 162:925-42, 2005.

[7] Shastry B S. “Pharmacogenetics and the concept of individualized medicine”, J. Pharmacogenomics, 6:16-21, 2006.

[8] Tenover F C. “Diagnostic Deoxyribonucleic Acid Probes for Infectious Diseases”, Clin. Microbiol. Rev., 1:82-101, 1988.

[9] McLaughlin G L, Ruth J L, Jablonski E, Steketee R, Campbell G H. “Use of enzyme-linked synthetic DNA in diagnosis of falciparum malaria”, Lancet, 329 (8535):714-716, 1987.

[10] Sauls C D, Caskey C T. “Applications of Recombinant DNA to Pathologic Diagnosis”, Clin. Chem., 31:804-811, 1985.

[11] Vosberg H P. “The polymerase chain reaction: an improved method for the analysis of nucleic acids”, Hum. Genet., 83:1-15, 1989.

[12] Kirk B W, Feinsod M, Favis R, Kliman R M, Barany F. “Single nucleotide polymorphism seeking long term association with complex disease”, Nucleic Acids Res., 30:3295-3311, 2002.

[13] Whelen A C, Persing D H. “The role of nucleic acid amplification and detection in the clinical microbiology laboratory”, Annu. Rev. Microbiol., 50:349-373, 1996.

[14] Bernard P S, Wittwer C T. “Real-Time PCR technology for cancer diagnostics”, Clin. Chem., 48:1178-1185, 2002.

[15] Mackay I M, Arden K E, Nitsche A. “Real-time PCR in virology”, Nucleic Acids Res., 30:1292-1305, 2002

[16] Siitari H. “Nucleic acid diagnostics market—Unmet needs and product potential”, National Technology Agency, Technology Review 125/2002, Helsinki, 2002.

[17] Twyman R M. “SNP Discovery and Typing Technologies for Pharmacogenomics”, Curr. Top. Med. Chem., 4:1421-1429, 2004

[18] Storhoff J J, Lucas A D, Garimella V, Bao Y P, Muller U R. “Homogeneous detection of unamplified genomic DNA sequences based on colorimetric scatter of gold nanoparticle probes”, Nat. Biotechnol., 22:883-887, 2004.

[19] Storhoff J J, Marla S S, Bao Y P, Hagenow S, Mehta H, Lucas A D, Garimella V, Patno T, Buckingham W, Cork W, Muller U R. “Gold nanoparticle-based detection of genomic DNA targets on microarrays using a novel optical detection system”, Biosensors and Bioelectronics, 19:875-883, 2004.

[20] Batista P, Doria G, Henriques D, Pereira E, Franco R. “Colorimetric detection of eukaryotic gene expression with DNA-derivatized gold nanoparticles”, Journal of Biotecnology, 119:111-117, 2005.

[21] Cao Y C, Jin R, Thaxton C S, Mirkin C A. “A two-color-change, nanoparticle-based method for DNA detection”, Talanta, 67:449-455, 2005.

[22] Storhoff J J, Elghanian R, Mucic R C, Mirkin C A, Letsinger R L. “One-pot colorimetric differentiation of polynucleotides with single base imperfections using gold nanoparticle probes”, J. Am. Chem. Soc., 120:1959-1964, 1998.

[23] Mirkin C A, Letsinger R L, Mucic R C, Storhoff J J. “A DNA-based method for rationally assembling nanoparticles into macroscopic materials”, Nature, 382:607-609, 1996.

[24] Sato K, Hosokawa K, Maeda M. “Non-cross-linking gold nanoparticle aggregation as a detection method for single-base substitutions”, Nucleic Acids Res, 33 (1):e4, 2005.

[25] Baptista P V, Koziol-Montewka M, Paluch-Oles J, Doria G, Franco R. “Gold nanoparticle probe-based diagnostic system for rapid and sensitive detection of Mycobacterium tuberculosis”, Clin Chem., 52:1433-1434, 2006.

[26] Faustino P, Osório-Almeida L, Barbot J, Espirito-Santo D, Gonçalves J, Romão L, Martins M C, Marques M M, Lavinha J. “Novel promoter and splice junction defects add to the genetic, clinical or geographic heterogeneity of beta-thalassaemia in Portuguese population”, Hum. Genet., 89:573-576, 1992

1. A colorimetric method for the detection of specific nucleic acids sequences, including mutations or single nucleotide polymorphisms in nucleic acids sequences, comprising inducing aggregation of nanoparticles functionalized with modified oligonucleotides by increasing the ionic strength of the medium, also including a blank assay, a positive control assay, a negative control assay and one or more assays with the target nucleic acid sample, being that each assay includes a denaturation step, a hybridization step, a development step and a result recording step, sequentially by this order.

2. The colorimetric method of claim 1, wherein the nanoparticles are made of metal, including at least one of gold, silver, gold/silver alloy and an alloy of gold with another metal, or a combination of one or more of these nanoparticles.

3. The colorimetric method of claim 1, wherein the nanoparticles are spherical, cylindrical, triangular, cubic, prismatic, with a rod shape, or with any other geometrical shape, solid or hollow, and with dimensions between 1 and 200 nm.

4. The colorimetric method of claim 1, wherein the nanoparticles are functionalized with DNA or RNA oligonucleotides with 10 to 100 nucleotides and modified with a thiol group at the 3′ or 5′ end, through which they bind to the nanoparticles by a quasi-covalent bond.

5. The colorimetric method of claim 1, wherein the nanoparticles are dispersed in distilled water or in a phosphate, citrate, Tris, Hepes (4-(2-hydroxyethyl)-1-piperazineethanesulfonic acid), TAPS (N-Tris(hydroxymethyl)methyl-3-aminopropanesulfonic acid), MOPS (3-(N-morpholino)propanesulfonic acid), PIPES (Piperazine-1,4-bis(2-ethanesulfonic acid) buffers, hypersolutes (for example, mannosylglycerate) or any other buffer with a pH between 1 and 14.

6. The colorimetric method of claim 5, wherein the buffer contains one, or more, salts, such as NaCl, MgCl₂, NiCl₂, NaBr, ZnCl₂, MnCl₂, BrCl, CaCl₂, KCl, AgCl, LiBr, KBr, AgBr, or other, in a final concentration between 0 and 2 M.

7. The colorimetric method of claim 4, wherein the oligonucleotides have one, or more, alkyl groups containing one, two, three, four, five, six, seven, eight, nine, ten, eleven, twelve, or more carbons, between the thiol group and the nucleotides.

8. The colorimetric method of claim 7, wherein the oligonucleotides are constituted by a specific sequence, or a combination of one, or more, different specific sequences, each one harbouring one sequence complementary to the nucleic acid target sequence, or one, or more, nucleotides non-complementary to the nucleic acid target sequence, between the 3′ and 5′ ends of the oligonucleotide.

9. The colorimetric method of claim 8, wherein the non-complementary nucleotides are related to single nucleotide polymorphisms, single point mutations, or any other type of mutation in an animal, human or pathogen genome.

10. A The colorimetric method of claim 4, wherein the blank assay includes a solution containing functionalized nanoparticles.

11. The colorimetric method of claim 1, wherein the positive control assay includes a solution containing the functionalized nanoparticles, as claimed in claim 4, and a nucleic acid complementary to the sequence functionalized in the same nanoparticles.

12. The colorimetric method of claim 1, wherein the negative control assay includes a solution containing functionalized nanoparticles and a nucleic acid non-complementary to the sequence functionalized in the same nanoparticles.

13. The colorimetric method of claim 1, wherein the denaturation step consists of denaturation of the nucleic acids present in solution using temperature, denaturing agents, pH or enzymes, or a combination of these.

14. The colorimetric method of claim 4, wherein the hybridization step consists in of the hybridization of the oligonucleotides functionalized on the nanoparticles with the respective complementary sequences of the nucleic acid targets present in solution, for a period between 0 minutes and 24 hours, or more, and at room temperature, or between 4° C. and 80° C.

15. The colorimetric method of claim 14, wherein the hybridization step includes the use of a hybridization buffer, such as phosphate, citrate, Tris, Hepes (4-(2-hydroxyethyl)-1-piperazineethanesulfonic acid), TAPS (N-Tris(hydroxymethyl)methyl-3-aminopropanesulfonic acid), MOPS (3-(N-morpholino)propanesulfonic acid), PIPES (Piperazine-1,4-bis(2-ethanesulfonic acid) buffers, hypersolutes (for example, mannosylglycerate) or any other buffer solution, containing, or not, denaturing agents, salts, inert polymers, surfactants, among others.

16. The colorimetric method of claim 1, wherein the development step consists of adding an electrolyte to the hybridization solution, such as, for example, NaCl, MgCl₂, NiCl₂, NaBr, ZnCl₂, MnCl₂, BrCl, CdCl₂, CaCl₂, CoCl₂, CoCl₃, CuCl₂, CuCl, PbCl₂, PtCl₂, PtCl₄, KCl, RbCl, AgCl, SnCl₂, BrF, LiBr, KBr, AgBr, NaNO₂, Na₃PO₄, Na₂HPO₄, NaH₂PO₄, KH₂PO₄, K₂HPO₄, among others, to a final concentration between 0 and 6 M, or more, during between 0 minutes and 1 hour, or more.

17. The colorimetric method of claim 1, wherein the result recording step consists in observing by the naked eye the colorimetric changes of different assays or by measuring the assays by absorption spectroscopy in the visible, ultra-violet or infrared regions.

18. The colorimetric method of claim 17, wherein the result registration step the initial colour of the assays is kept unchanged indicating the presence of one, or more, nucleic acid targets that are complementary to the functionalized nanoparticles.

19. The colorimetric method of claim 17, wherein the result recording step the initial colour of the assays changes indicating the presence of one, or more, nucleic acid targets that are not complementary to the functionalized nanoparticles or the presence of one mutation or single nucleotide polymorphism in the target nucleic acid sequence, or yet the absence of any nucleic acid target in solution.

20. The colorimetric method of claim 17, wherein in the result recording step, the ratio between the initial absorption peak and the peak after the development step of the functionalized nanoparticles constitutes a quantifying measure of the nucleic acid targets in solution, or a way to identify a mutation or a single nucleotide polymorphism in the sequence of the nucleic acid target.

21. The colorimetric method of claim 1, wherein the nucleic acid target is genomic DNA, single stranded DNA, double stranded DNA, plasmid, cosmid, BAC, YAC, HAC, total RNA, messenger RNA, ribosomal RNA, transfer RNA, synthesized DNA, synthesized RNA, among others.

22. The colorimetric method of claim 21, wherein the nucleic acid target is previously enzymatically amplified by PCR or an isothermal reaction such as LAMP (loop-mediated isothermal amplification), among others.

23. Use of a colorimetric method, as claimed in claim 1 wherein it is used to detect specific nucleic acid sequences through the use of metal nanoparticles functionalized with modified oligonucleotides.

24. A kit for the detection of specific nucleic acid sequences, including mutation or single nucleotide polymorphisms in sequences of nucleic acids, accordingly to the method claimed in claim 1, wherein it contains:
a) one or more solutions that include the functionalized nanoparticles;
b) a solution of hybridization buffer;
c) a development solution;
d) one or more control solutions containing nucleic acids that are complementary and/or non-complementary to the sequences in the functionalized nanoparticles.
that are deposited in a microplate or on a microfluidic system, or yet in any other type of container suitable to the execution of the method claimed in claim 1.

25. Use of the kit for the detection of specific nucleic acid sequences, as claimed in claim 1, wherein it is used to detect specific nucleic acid sequences, including mutations or single nucleotide polymorphisms, in the sequences of nucleic acids, accordingly to the method claimed in claim 1 to 22.
